(19) United States
(12) Patent Application Publication
(10) Pub. No.: US 2020/0010394 A1
(43) Pub. Date: Jan. 9, 2020

(54) METHOD FOR PREPARING 1-HYDROXY-2-METHYL-3-PENTANONE

(71) Applicant: BASF SE, Ludwigshafen am Rhein (DE)

(72) Inventors: Dagmar Pascale KUNSMANN-KEITEL, Ludwigshafen am Rhein (DE); Georg Heinrich GROSCH, Ludwigshafen am Rhein (DE); Jan-Dirk ARNDT, Ludwigshafen am Rhein (DE); Gauthier Luc Maruice AVERLANT, Darmstadt (DE); Wolf-Steffen WEISSKER, Ludwigshafen am Rhein (DE); Marcus Georg SCHREMS, Ludwigshafen am Rhein (DE); Dan-Tam Daniel TANG, Ludwigshafen am Rhein (DE)

(21) Appl. No.: 16/460,327

(22) Filed: Jul. 2, 2019

(30) Foreign Application Priority Data
Jul. 6, 2018 (EP) 18182103.4

(51) Int. Cl. C07C 45/64; C07C 45/83; C07C 49/17

(52) U.S. Cl. CPC C07C 45/64; C07C 49/17; C07C 45/83

(57) ABSTRACT

Method for preparing 1-hydroxy-2-methyl-3-pentanone (I) by reacting formaldehyde with diethyl ketone in a reactor in the presence of water and a basic component at a temperature of 50 to 150° C. and a pressure of 0.2 to 10 MPa abs, in which the basic component used is a trialkylamine from the group comprising trimethylamine, N,N-dimethylethylamine, N,N-diethylmethylamine, triethylamine, N,N-dimethyl-n-propylamine, N-ethyl-N-methyl-n-propylamine, N,N-dimethylisopropylamine, N-ethyl-N-methylisopropylamine, N,N-dimethyl-n-butylamine, N,N-dimethylisobutylamine and N,N-dimethyl-sec-butylamine, and from the reaction mixture obtained, trialkylamine as low boiler and a bottom product comprising 1-hydroxy-2-methyl-3-pentanone (I) as high boiler are separated in a distillation apparatus, wherein the distillation apparatus is operated at a top pressure of 0.2 to 1 MPa abs.

### CROSS-REFERENCE TO RELATED APPLICATIONS

This application claims priority to European Application No. 18182103.4, filed Jul. 6, 2018, which is incorporated herein by reference in its entirety.

The present invention relates to a method for preparing 1-hydroxy-2-methyl-3-pentanone by reacting formaldehyde with diethyl ketone in a reactor in the presence of water and a basic component at a temperature of 50 to 150° C. and a pressure of 0.2 to 10 MPa abs and subsequent distillation thereof.

1-hydroxy-2-methyl-3-pentanone can be used as a solvent, owing to its polar properties, for example for natural or synthetic resin varnishes. Due to its hydroxyl and keto group, 1-hydroxy-2-methyl-3-pentanone is also of interest as a synthetic unit for the preparation of active ingredients. It can also be converted to the corresponding enone by elimination of water, which likewise is an interesting synthetic unit.

From a reaction engineering perspective, aldol addition of formaldehyde to diethyl ketone provides the industrial synthesis of 1-hydroxy-2-methyl-3-pentanone.

Aldol additions are well-known to those skilled in the art. In aldol addition, aldehydes and ketones react to give β-hydroxyaldehydes or β-hydroxyketones. They can be be carried out base-catalyzed or acid-catalyzed. In the case of a base-catalyzed aldol addition, an enolate anion is initially formed by base-catalyzed H⁺ elimination at the α-position, which in the form of its nucleophilic methylene component then adds to the second carbonyl component with addition of H⁺ and recovery of the basic component.

For the synthesis of 1-hydroxy-2-methyl-3-pentanone (I), the base-catalyzed aldol addition of formaldehyde (II) to diethyl ketone (III) appears advisable from a reaction engineering perspective, because the basic catalyst can be selected by using a tertiary amine such that the latter can be easily removed as low boiler by subsequent distillation and can be recycled. The reaction equation using a trialkylamine as catalyst is therefore

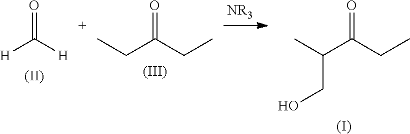

wherein NR₃ is a trialkylamine with the same or different alkyl radicals. Typically, the reaction would be operated at elevated temperature (above room temperature) and elevated pressure (above atmospheric pressure).

A typical secondary reaction in the aldol addition of formaldehyde to ketones is the multiple addition of formaldehyde. For instance, by-products formed in the synthesis of 1-hydroxy-2-methyl-3-pentanone (I) by two-fold addition are 1,5-dihydroxy-2,4-dimethyl-3-pentanone (IV) and 1-hydroxy-2-hydroxymethyl-2-methyl-3-pentanone (V) and formed as by-product by three-fold addition is 1,5-dihydroxy-2-hydroxymethyl-2,4-dimethyl-3-pentanone (VI).

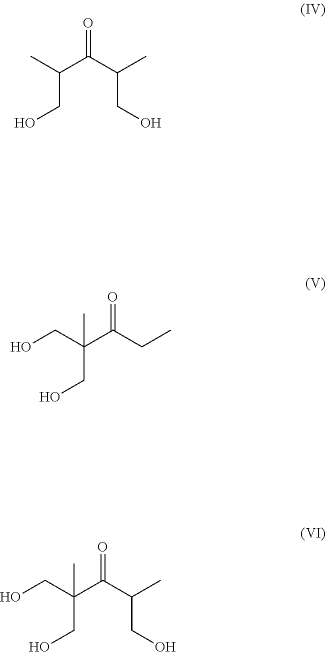

Furthermore, β-hydroxyketones such as also the compounds (I), (IV), (V) and (VI) for example, generally tend to eliminate water to form an α,β-unsaturated ketone (enone) in the presence of acids or bases in the case of supply of heat.

For instance, the textbook Organic Chemistry: Structure, Mechanism, and Synthesis, Robert J. Ouellette and J. David Rawn, Elsevier 2014, ISBN 978-0-12-800780-8 in chapter 22.6 describes the base-catalyzed aldol condensation of aldehydes with elimination of water to give the corresponding enals. In a first step, the addition product is initially formed, which then dehydrates at elevated temperature to the enal. In addition to the dehydration in the presence of a catalytic amount of a base, the dehydration can also be catalyzed by strong acids.

The textbook Organic Chemistry, John McMurry, 5th edition, Brooks/Cole 2000, ISBN 0-534-37366-6 in chapter 23.4 describes the dehydration of β-hydroxyaldehydes and 3-hydroxyketones to the corresponding enals and enones. The dehydration can be base-catalyzed with elimination of an acidic α-hydrogen and subsequent elimination of an OH⁻ group, and acid-catalyzed with protonation of the ═O group and also the OH group, elimination of an α-hydrogen and subsequent elimination of an H₃O⁺ group. The document teaches that somewhat more forceful conditions are often required, such as a somewhat elevated temperature, than in the preceding aldol addition.

Both textbooks therefore teach that, in addition to catalytically active amounts of a base or an acid, the formation of enals and enones from β-hydroxyaldehydes and β-hydroxyketones particularly require elevated temperatures. For instance, the enone 2-methyl-1-penten-3-one (VII) can be formed by dehydration of 1-hydroxy-2-methyl-3-pentanone (I) and the enone 2,4-dimethyl-5-hydroxy-1-penten-3-one (VIII) can be formed by dehydration of 1,5-dihydroxy-2,4-dimethyl-3-pentanone (IV)

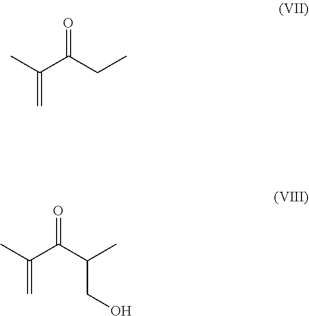

Since the enones formed are unsaturated reactive compounds, these tend to further reaction, such as to dimerization, oligomerization or even polymerization for example.

The reaction mixture obtained in the synthesis of 1-hydroxy-2-methyl-3-pentanone (I) by the aldol addition specified therefore comprises, in addition to the target product (I), a whole series of further components such as water, trialkylamine, unreacted formaldehyde and/or unreacted diethyl ketone, as well as diverse lower-boiling and higher-boiling by-products, which are to be subsequently removed to obtain the purest possible 1-hydroxy-2-methyl-3-pentanone (I).

In the work-up of such a complex composite mixture, a fractional or multi-stage distillation is typically used for this purpose, in which the low boilers and high boilers are removed in stages from the 1-hydroxy-2-methyl-3-pentanone (I). The known tendency of β-hydroxyketones to dehydrate to α,β-unsaturated ketones requires caution however.

For instance, U.S. Pat. No. 3,077,500 shows that, in the case of distillation of 4-hydroxy-3-methyl-2-butanone at atmospheric pressure in the presence of phosphoric acid, virtually complete dehydration of the β-hydroxyketone takes place despite the relatively low temperatures. As is known from the textbooks cited, acids strongly promote dehydration.

U.S. Pat. No. 3,077,500 describes in example X the aldol addition of formaldehyde to methyl ethyl ketone in the presence of triethylamine at a pressure of 0.31 MPa abs (45 psi). The reaction mixture was passed continuously through the reactor in this case, methyl ethyl ketone was added, and fed as return to the top of a distillation apparatus operated at atmospheric pressure. The vapor stream of the distillation apparatus was condensed, aqueous formaldehyde solution and triethylamine were added and pumped into the reactor under pressure. The aldol addition product 4-hydroxy-3-methyl-2-butanone was withdrawn continuously from the bottom of the column, phosphoric acid was added and transferred to a second distillation apparatus for the dehydration. This was also operated at atmospheric pressure. Finally, the enones 3-methyl-3-buten-2-one and 1-penten-3-one were formed under the conditions stated in a yield of 86.6%.

U.S. Pat. No. 3,662,001 teaches the fractional distillation under vacuum of the reaction mixture formed by aldol addition of formaldehyde to acetone in the synthesis of 3-keto-1-butanol. As a result, thermal stress can be kept low.

The formation of enones is disadvantageous and therefore undesirable for several reasons.
1. Firstly, the dehydration of 1-hydroxy-2-methyl-3-pentanone (I) to 2-methyl-1-penten-3-one (VII) results in irreversible loss of product of value.
2. Secondly, any increase of enones generally leads to an increased risk that undesired deposits form on the apparatuses due to oligomeric or polymeric by-products and these have to be cleaned from time to time.
3. Thirdly, the formation of enones increases the complexity of the work-up of the reaction mixture since these and conversion products thereof of 1-hydroxy-2-methyl-3-pentanone (I) are to be removed as low boilers or high boilers.
4. Fourthly, it is recognized very specifically in accordance with the invention that 2,4-dimethyl-5-hydroxy-1-penten-3-one (VIII) having a boiling point of 209° C. at 0.1 MPa abs (or of ca. 233° C. at 0.2 MPa abs) can only be separated with great difficulty by distillation due to its boiling point from 1-hydroxy-2-methyl-3-pentanone (I), which has a boiling point of 202° C. at 0.1 MPa abs (or of ca. 231° C. at 0.2 MPa abs).

It is therefore desirable in the synthesis and distillative work-up of 1-hydroxy-2-methyl-3-pentanone (I) to use process conditions which do not excessively promote or even substantially prevent the dehydration of the β-hydroxyketones to the corresponding enones. Accordingly, the prior art cited suggests a particularly mild work-up of the reaction mixture, particularly with regard to thermal stress. Specifically, the prior art teaches conducting the distillative work-up under reduced pressure.

It was the object of the present invention to find a method for preparing 1-hydroxy-2-methyl-3-pentanone (I), which is based on readily accessible feedstocks, is simple and safe to carry out, enables the highest possible selectivity, yield and purity of product of value, and in particular largely avoids or at least significantly reduces the formation of undesired by-products with reduction of the yield, increase in the amounts used and increase in complexity for the separation and disposal. In particular, the method according to the invention should keep the formation of 2,4-dimethyl-5-hydroxy-1-penten-3-one (VIII) as low as possible, but at the same time keep the outlay in terms of apparatus within limits in the context of an efficient process regime.

Surprisingly, a method for preparing 1-hydroxy-2-methyl-3-pentanone (I) has been found by
(a) reacting formaldehyde (II) with diethyl ketone (III) in a reactor (A) in the presence of water and a basic component at a temperature of 50 to 150° C. and a pressure of 0.2 to 10 MPa abs,

in which

the basic component used is a trialkylamine from the group comprising trimethylamine, N,N-dimethylethylamine, N,N-diethylmethylamine, triethylamine, N,N-dimethyl-n-propylamine, N-ethyl-N-methyl-n-propylamine, N,N-dimethylisopropylamine, N-ethyl-N-methylisopropylamine, N,N-dimethyl-n-butylamine, N,N-dimethylisobutylamine, N,N-dimethylsec-butylamine and
(b) from the reaction mixture obtained, trialkylamine as low boiler and a bottom product comprising 1-hydroxy-2-methyl-3-pentanone (I) as high boiler are separated in a distillation apparatus (B), wherein the distillation apparatus (B) is operated at a top pressure of 0.2 to 1 MPa abs.

### A BRIEF DESCRIPTION OF THE FIGURES

FIG. 1 illustrates an exemplary representation of a continuously operated plant according to the invention.

FIG. 2 shows the increase of components (VII) and (VIII) over the heat treatment time in graphic form.

FIG. 3 shows the increase of components (VII) and (VIII) over the heat treatment time in graphic form.

FIG. 4 shows the time profile of components (VII) and (VIII) via the various settings, wherein the time scale here is presented continuously from day 1 to day 25.

In the method according to the invention for preparing 1-hydroxy-2-methyl-3-pentanone (I), formaldehyde (II) is reacted with diethyl ketone (III) in a reactor (A) in the presence of water and a trialkylamine from the group comprising trimethylamine, N,N-dimethylethylamine, N,N-diethylmethylamine, triethylamine, methyl-n-propylamine, N-ethyl-N-methyl-n-propylamine, N,N-dimethylisopropylamine, N-ethyl-N-methylisopropylamine, N,N-dimethyl-n-butylamine, N,N-dimethylisobutylamine and N,N-dimethyl-sec-butylamine as basic component at a temperature of 50 to 150° C. and a pressure of 0.2 to 10 MPa abs. In the reaction equation below, NR₃ is the corresponding trialkylamine.

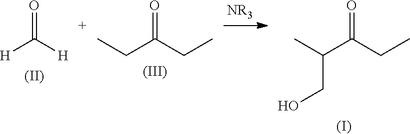

As already explained at the outset, the aldol addition does not necessarily stop at the target product 1-hydroxy-2-methyl-3-pentanone (I), but continues to some extent by further additions of formaldehyde (II) to give the corresponding di- and trihydroxy compounds 1,5-dihydroxy-2,4-dimethyl-3-pentanone (IV), 1-hydroxy-2-hydroxymethyl-2-methyl-3-pentanone (V) and 1,5-dihydroxy-2-hydroxymethyl-2,4-dimethyl-3-pentanone (VI). The β-hydroxyketo group is common to the target product 1-hydroxy-2-methyl-3-pentanone (I) and the di- and trihydroxy compounds. These can lead by dehydration to the corresponding enone, which as reactive molecules in turn have a tendency to dimerization and further secondary reactions. Accordingly, there is thus diversity in the typical by-product spectrum in the aldol addition specified.

In order to keep the multiple addition of formaldehyde (II) to diethyl ketone (III) to a level as low as possible, an excess of diethyl ketone (III) is generally used in the reaction in step (a). The molar ratio of diethyl ketone (III) to formaldehyde (II) in the liquid phase is preferably from 1.5 to 10, preferably≥2 and particularly preferably≥3, and preferably≤7 and particularly preferably≤5.

In the reaction in step (a), different qualities and forms of formaldehyde can be used. For instance, it is possible to also feed pure formaldehyde to the reactor. The use of paraformaldehyde is also possible in principle, although not particularly preferred. Typically however, easier to handle formaldehyde sources are used, such as especially its aqueous solutions. These may be of technical-grade quality and laboratory quality. The formaldehyde content of aqueous solutions is typically in the range from 30 to 60% by weight. For instance, in the method according to the invention, the formaldehyde (II) is preferably used in the form of its aqueous solutions having a formaldehyde content of 30 to 60% by weight.

Aqueous solutions of formaldehyde also typically comprise methanol as stabilizer. Since formaldehyde is commonly obtained by catalytic oxidation of methanol, the methanol present is generally wholly or partly unreacted methanol from the formaldehyde synthesis. Depending on the quality of the formaldehyde solution, the methanol content can vary in a wide range. Generally therefore, in the reaction in step (a), an aqueous formaldehyde solution is used having a methanol content of 0.1 to 20% by weight, preferably 0.2 to 5% by weight.

In general, in the reaction in step (a), an aqueous methanol-containing solution of formaldehyde is used as formaldehyde source.

In the method according to the invention, the basic component used is a trialkylamine from the group comprising trimethylamine, N,N-dimethylethylamine, N,N-diethylmethylamine, triethylamine, N,N-dimethyl-n-propylamine, N-ethyl-N-methyl-n-propylamine, N,N-dimethylisopropylamine, N-ethyl-N-methylisopropylamine, N,N-dimethyl-n-butylamine, N,N-dimethylisobutylamine and N,N-dimethylsec-butylamine. The trialkylamines specified have the advantage that these can be removed as low boilers by distillation from diethyl ketone (III) as required.

Among the alkylamines mentioned, particularly advantageous is the use of trimethylamine and N,N-dimethylethylamine since these can be removed as low boilers by distillation as required, even from methanol. Particular preference is given to using trimethylamine.

The amount of trialkylamine used in the reaction in step (a) can be varied within a wide range. Since trialkylamine functions as a catalyst, it is also therefore not consumed. Generally, a molar ratio of trialkylamine to formaldehyde (II) in the liquid phase is used from 0.001 to 10. A very low molar ratio of only 0.001, however, results in a low space-time yield such that it is advantageous for this reason to use a molar ratio of trialkylamine to formaldehyde (II) in the liquid phase of >0.01 or even>0.1. A corresponding molar ratio of 0.01 to 10, and particularly preferably of 0.1 to 10, is therefore preferable. In addition, surprisingly, it has been shown that at a molar ratio of trialkylamine to formaldehyde (II) in the liquid phase of 1 to 5, significantly fewer undesirable by-products are formed than when using a substantially lower molar ratio of, for example, less than 0.4.

Very particular preference is therefore given to a corresponding molar ratio of 1 to 5, particularly of≥1.1 and especially of≥1.2 and also particularly of≤3 and especially of≤2.

In addition to the two reactants formaldehyde (II) and diethyl ketone (III) and the trialkylamine functioning as catalyst, water is also used as further component in the reaction in step (a). In general, the reaction specified is carried out in the presence of 1 to 25% by weight water, based on the liquid phase. This is typically added via the formaldehyde source, i.e. an aqueous formaldehyde solution, via a separate addition of water or in parallel by both possibilities. In the presence of trialkylamine, the presence of water enables the formation of trialkylammonium hydroxide, which is the active part of the catalyst. The amount of water therefore influences the reaction rate. With increasing amount of water, the reaction rate also generally increases. However, greater amounts of water results in enhanced formation of undesired by-products. Thus, the reaction in step (a) is preferably carried out in the presence of≥5% by weight, particularly preferably≥8% by weight and especially preferably≥10% by weight, and preferably in the presence of≤17% by weight and particularly preferably≤15% by weight, based in each case on the liquid phase. In particular, the reaction is carried out in the presence of 10 to 15% by weight water, based on the liquid phase.

If an aqueous methanol-containing formaldehyde solution is used as formalde-hyde source in the reaction in step (a), the reaction in step (a) is inevitably also carried out in the presence of methanol. Independently thereof, methanol can however also be added to the reaction separately. Furthermore, it is possible and possibly even advantageous to recycle into the reactor the methanol present in the reaction mixture, together with other components such as unreacted diethyl ketone (III), water and/or trialkylamine, in the context of the work-up of the reaction mixture.

In a preferred embodiment, therefore, the reaction is carried out in the presence of methanol. The reaction in step (a) is generally carried out in the presence of 0.1 to 10% by weight methanol, based on the liquid phase. Methanol contents above 10% by weight result in a noticeable slowing of the reaction. The reason is probably the enhanced formation of acetals from formaldehyde and methanol, which results in loss of freely available formaldehyde. Thus, the reaction in step (a) is preferably carried out in the presence of≤6% by weight and particularly preferably≤3% by weight methanol, and preferably in the presence of≥0.1% by weight and particularly preferably≥0.5% by weight methanol, based in each case on the liquid phase.

The reaction in step (a) is carried out at a temperature of 50 to 150° C., preferably at≥55° C., particularly preferably at 60° C. and especially preferably at≥65° C., and preferably at≤125° C. and particularly preferably at≤100° C. In addition, the reaction in step (a) is carried out at a pressure of 0.2 to 10 MPa abs, preferably≤0.3 MPa abs and particularly preferably≥0.4 MPa abs, and preferably≤2 MPa abs, particularly preferably≤3 MPa abs and especially preferably≤2 MPa abs.

According to the temperature and pressure parameters specified, the reaction mixture is completely, or at least to a considerable proportion, present in the liquid phase.

The reaction in step a) can be carried out in batchwise mode, continuously or semi-continuously.

In the batchwise variant, the reactants formaldehyde (II) and diethyl ketone (III), together with trialkylamine, water and optionally further components such as methanol for example, are initially charged in the desired amounts in the reactor and these are subsequently brought to the desired temperature and the desired pressure with appropriate mixing of the reaction mixture for the desired reaction time. After the desired reaction time, the reaction mixture is withdrawn for further processing, optionally after completion of depressurization and cooling.

In the continuous variant, the reactants formaldehyde (II) and diethyl ketone (III), together with trialkylamine, water and optionally further components, such as methanol for example, are fed continuously to the reactor in the desired amounts and, at suitable points, a corresponding amount of reaction mixture is also continuously withdrawn. The reaction mixture is accordingly reacted in the reactor at the desired temperature and the desired pressure. The residence time in the continuous method is dependent on the desired reaction progress. The reaction mixture continuously withdrawn can then be worked-up accordingly.

In the semi-continuous variant, typically diethyl ketone (III), together with trialkylamine and optionally water and further components, such as methanol for example, are initially charged in the desired amounts in the reactor, these are then brought with appropriate mixing to the desired temperature and the desired pressure and the desired amount of formaldehyde (II) and optionally water and methanol are then fed over a certain time period. After the desired reaction time, the reaction mixture is withdrawn for further processing, optionally after completion of depressurization and cooling.

The reaction in step (a) in the method according to the invention is preferably carried out continuously.

Since the aldol addition is mildly exothermic, heat of reaction is formed in the reaction in step (a). This can be dissipated in adiabatic mode via the reaction output. In isothermal mode of operation, this is already dissipated in the reactor during the reaction and the reaction temperature stays constant. In the method according to the invention, in principle both variants and all in-between stages are possible. In principle, variants are also possible in which, for example, thermal energy is supplied externally and the reaction mixture heats up more intensely than in adiabatic mode, or for example in which more thermal energy is removed than is required for an isothermal mode of operation, the reaction mixture thus being cooled during the reaction.

The reaction in step (a) takes place in reactor (A). In principle, any apparatuses can be used as reactor (A) which are suitable for carrying out reactions in the liquid phase. Non-limiting examples include flow tube, jet loop reactor, stirred tank and stirred tank cascade and combinations thereof. Depending on whether the reaction is conducted in batchwise mode, continuously or semi-continuously, particular reactors have proven to be particularly advantageous. For instance, in a particularly preferred variant, the method is carried out continuously in a flow tube and in another preferred variant, the method is carried out batchwise or semicontinuously in a stirred tank. A combination of one or more stirred tanks or jet loop reactors as preliminary reactor(s) and one or more flow tubes as postreactor(s) is also possible. In such a case, the overall combination is referred to as reactor (A).

In accordance with the selected residence time of the reaction mixture in the reactor, the conversion of the reaction mixture can be adjusted. At a fairly low residence time of less than one hour, the conversion is generally still incomplete such that all of the formaldehyde (II) is typically still not reacted and is subsequently to be removed. Conversely, if the residence time is close to one day or even longer, the amount of undesired by-products also generally increases. In addition, a long residence time also reduces the space-time yield.

In the batchwise reaction, the reactor is thus typically left at the desired reaction conditions for 1 to 16 hours, preferably≥1.5 and particularly preferably≥2 hours, and preferably≤10 and particularly preferably≤6 hours.

Accordingly, in the continuous reaction, the flow volume entering the reaction system is adjusted so that a residence time results of typically 1 to 16 hours, preferably≥1.5 and particularly preferably≥2 hours, and preferably≤10 and particularly preferably≤6 hours at the desired reaction conditions.

The semi-continuous process is modelled on the batchwise reaction with respect to the reaction time.

The reaction mixture obtained in step (a) in particular comprises the 1-hydroxy-2-methyl-3-pentanone (I) formed, unreacted diethyl ketone (III), trialkylamine, water and by-products formed, such as di- and trihydroxy compounds formed by multiple addition for example, and possibly even enones already formed by dehydration. If methanol was also present in the reaction, which is typically the case when preferably using an aqueous formaldehyde solution, the reaction mixture also comprises methanol.

Typically, the reaction mixture from the reaction in step (a) comprises a content of 1-hydroxy-2-methyl-3-pentanone (I) of only 5 to 25% by weight. Owing to the preferred excess of diethyl ketone (III) in the preceding reaction in step (a), the reaction mixture also typically comprises an appreciable amount of diethyl ketone (III) at a similar order of magnitude or even significantly more. In general, the reaction mixture obtained in step (a) is composed as follows, wherein the last column gives the preferred range:

| | Content [% by wt.] | |
|---|---|---|
| Component | In general | Preferred |
| 1-Hydroxy-2-methyl-3-pentanone | 5-25 | 10-20 |
| (I) | | |
| Formaldehyde (II) | 0-1 | 0-0.1 |
| Diethyl ketone (III) | 5-75 | 30-60 |
| Trialkylamine | 0.001-20 | 5-20 |
| Water | 5-20 | 10-18 |
| Methanol | 0-10 | 0.1-6 |
| Dihydroxy compounds (IV) + (V) | 0.5-8 | 1-5 |
| Trihydroxy compound (VI) | 0.1-1 | 0.2-0.5 |
| Enone (VII) | 0.1-1 | 0.2-0.5 |
| Enone (VIII) | 0.1-1 | 0.2-0.5 |

It is self-evident that the sum total of the contents specified cannot exceed 100% by weight. Since in the reaction mixture, besides the components explicitly mentioned, further by-products may also be present, such as oligomers or polymers of the enones, the sum total of the components specified can also be less than 100% by weight. The sum total specified is typically in the range from 95 to 100% by weight.

For the work-up of the reaction mixture, it is convenient to initially remove the low boilers by distillation and thus to concentrate the 1-hydroxy-2-methyl-3-pentanone (I) product of value.

However, since distillations are typically accompanied by thermal treatment of the mixture to be distilled, there exists in principle the risk of temperature-dependent or temperature-induced secondary reactions due to the thermal stress in the distillation apparatus. Specifically, in the present distillation task, there is especially the risk of a temperature-related dehydration of the β-hydroxyketones, especially of the compounds (I), (IV), (V), (VI) and (VIII). The dehydration of β-hydroxyketones by thermal stress was already mentioned at the outset as an undesirable secondary reaction. In order to largely avoid a temperature-related dehydration of β-hydroxyketones, it is thus obvious to carry out the removal of the low boilers mentioned under the mildest possible temperature conditions. In process engineering terms, this ultimately means adjusting to the lowest possible pressure, primarily atmospheric pressure or reduced pressure. For instance, U.S. Pat. No. 3,662,001 also discloses the distillation under reduced pressure of the reaction mixture formed in the production of the β-hydroxyketone 3-keto-1-butanol.

The considerable disadvantages of the dehydration of β-hydroxyketones to enones have already been mentioned in the introduction. Of particular emphasis in this case is the particularly disruptive disadvantage of dehydration of 1,5-dihydroxy-2,4-dimethyl-3-pentanone (IV) to 2,4-dimethyl-5-hydroxy-1-penten-3-one (VIII). This has a boiling point of 209° C. at 0.1 MPa abs (or of ca. 233° C. at 0.2 MPa abs), which is only marginally above the boiling point of 1-hydroxy-2-methyl-3-pentanone (I) at 202° C. at 0.1 MPa abs (or of ca. 231° C. at 0.2 MPa abs). As a consequence, 2,4-dimethyl-5-hydroxy-1-penten-3-one (VIII) can only be removed with great difficulty in the further processing by distillation from the 1-hydroxy-2-methyl-3-pentanone (I) product of value. As a consequence, either more or less large residual amounts of enone (VIII) remain in the 1-hydroxy-2-methyl-3-pentanone (I), or expenditure in terms of apparatus increases due to a significant increase in the number of theoretical plates. A specific reduction of the formation of enone (VIII), for example by distillation at the lowest possible temperature and therefore at low pressure, thus promotes the production of pure 1-hydroxy-2-methyl-3-pentanone (I).

Distillative removal of the trialkylamine low boiler from the reaction mixture obtained in step (a) at low pressure is technically quite possible, but requires the use of an appropriately low temperature-controlled cooling medium in the condenser in order to condense the trialkylamine. For instance, the boiling point of trimethylamine at 0.1 MPa abs is 3° C. The cooling medium required for the condensation must therefore be temperature-controlled at a temperature of<3° C. For better heat exchange, the temperature of the cooling medium should logically be at least 10° C. lower than the condensation temperature of the corresponding alkylamine. Therefore, to condense trimethylamine at 0.1 MPa abs, a cooling medium having a temperature of ideally minus 7° C. would be required. Commonly, in order to achieve this temperature, the use of a refrigeration plant is generally required.

Surprisingly it has now been found that, in the removal by distillation of the trialkylamine from the reaction mixture obtained in step (a), the formation of 2,4-dimethyl-5-hydroxy-1-penten-3-one (VIII) only increases marginally on increasing the pressure in the distillation apparatus and thus increasing the distillation temperature. For instance, it has been found that, surprisingly, at a top pressure of 0.525 MPa abs, the amount of 2,4-dimethyl-5-hydroxy-1-penten-3-one (VIII) in the bottoms increases by only about 0.1% by weight compared to a procedure at a top pressure of 0.1 MPa abs. Owing to the known tendency to dehydration of β-hydroxyketones, a substantially greater increase would have been expected due to the increase in the temperature associated therewith.

The distillative removal of the trialkylamine from the reaction mixture obtained in step (a) at a significantly higher top pressure than 0.1 MPa abs has the critical advantage of a higher condensation temperature of the trialkylamine. For instance, the condensation temperature of trimethylamine at a top pressure of 0.325 MPa abs is already 39° C. and, at 0.525 MPa abs, 56° C. This has the critical advantage that at 0.325 MPa abs, a cooling medium is sufficient at a temperature of≤39° C., preferably≤29° C., and also at 0.525 MPa abs of≤56° C., preferably≤46° C. Such temperatures can usually be ensured by customary cooling water or even air cooling, such that a refrigeration plant is dispensed with. The method is therefore simpler in apparatus terms and more energy-efficient, both in regard to the apparatuses required and during operation.

Therefore, in the method according to the invention, trialkylamine as low boiler and a bottoms product comprising 1-hydroxy-2-methyl-3-pentanone (I) as high boiler are separated in step (b) from the reaction mixture obtained in step (a) in a distillation apparatus (B), wherein the distillation apparatus (B) is operated at a top pressure of 0.2 to 1 MPa abs.

In principle, any distillation apparatuses can be used as distillation apparatus (B) which are suitable for removing trialkylamines having a boiling point below the boiling point of diethyl ketone (III) from a mixture at a top pressure of up to 1 MPa abs. As for customary distillation apparatuses, in addition to the column body itself, distillation apparatus (B) naturally also has a device for heating the bottom of the column, typically referred to as bottoms reboiler, and also a condenser. The term distillation apparatus (B) is to be understood as the whole distillation unit including column body, bottom reboiler and condenser, as well as associated withdrawal lines and return lines. The column body typically comprises trays, structured packings or random packings, preferably trays. The number of theoretical plates is typically from 3 to 50, preferably≥6 and particularly preferably≥15, and also preferably≤40 and particularly preferably≤35. Generally, the feed is introduced in the lower third of the column body. The condensers used in the distillation apparatus (B) are especially air coolers as well as water-cooled condensers of any construction type, such as shell and tube or plate heat exchangers.

The distillation apparatus (B) is operated at a top pressure of 0.2 to 1 MPa abs, preferably at≤0.6 MPa abs, and also preferably at≥0.25 MPa abs and particularly preferably at≥0.3 MPa abs.

The stream comprising trialkylamine passed into the condenser in the distillation apparatus (B) via the overhead is condensed therein and a portion of the condensate is fed back to the column as column return. Typically, the distillation apparatus (B) is operated at a return ratio of 2 to 15, preferably of≥3 and particularly preferably of≥4, and also preferably of≤10 and particularly preferably of≤8. The return ratio is understood to mean the quotient of the distillate fed back to the column and the distillate discharged from the distillation apparatus. The distillate comprising trialkylamine discharged from the distillation apparatus (B) can then be advantageously fed back wholly or partially to the reactor (A) for reuse.

Preference is given to a method in which trialkylamine is condensed in a condenser in distillation apparatus (B), removed from the distillation apparatus (B) and fed back to the reactor (A). Particularly preferably, 50 to 100% by weight and especially preferably 90 to 100% by weight, in particular the total amount of the trialkylamine withdrawn from the distillation apparatus (B), is fed back to the reactor (A).

If the reaction in step (a) is carried out in the presence of methanol, it is advantageous also to remove methanol as further low boiler in step (b). Since methanol is already fed to the reaction system together with formaldehyde (II) when using aqueous formaldehyde solution, but it is not chemically consumed, it is advantageous to discharge an appropriate amount of methanol in the form of a so-called purge stream in order to avoid accumulation.

In the presence of trimethylamine or N,N-dimethylethylamine, which are lower boiling than methanol, it is expedient firstly to condense the methanol and optionally a portion of the trimethylamine or N,N-dimethylethylamine in a first condenser at the top of the distillation apparatus (B), and then in a second condenser, which is operated at a lower temperature, to condense the residual trimethylamine or N,N-dimethylethylamine. The condensate of the second condenser is then fed back for reuse to the reactor (A), as already described two paragraphs above. The condensate of the first condenser comprising methanol is partially fed back to the column as return stream, and partially removed as so-called purge stream. The condensate is expediently divided such that, on the one hand the amount of condensate removed is large enough to avoid an accumulation of methanol, but also on the other hand the remaining return stream is sufficient to enable a reliable operation of the distillation apparatus (B).

Therefore, a method is preferred in which the reaction in step (a) is carried out in the presence of trimethylamine or N,N-dimethylethylamine as trialkylamine and methanol, and a two-stage condenser is used in the distillation apparatus (B) in step (b) for condensing the overhead product, and
(i) in the first condenser, a mixture comprising methanol is condensed and withdrawn from distillation apparatus (B), and
(ii) in the second condenser, a mixture comprising trimethylamine or N,N-dimethylethylamine is condensed, withdrawn from distillation apparatus (B) and is fed back to the reactor (A).

When using trimethylamine as trialkylamine, the condensed mixture in the first condenser preferably comprises 2 to 77% by weight methanol and the condensed mixture in the second condenser preferably comprises 59 to 98% by weight trimethylamine.

Particularly preferably, 50 to 100% by weight and especially preferably 90 to 100% by weight, in particular the total amount of the trimethylamine or N,N-dimethylethylamine condensed in the second condenser is fed back to the reactor (A).

In another preferred variant using trimethylamine or N,N-dimethylethylamine as trialkylamine, which are lower boiling than methanol, the desired amount of methanol as purge stream is withdrawn as sidestream from the rectifying section of the column in step (b). Trimethylamine or N,N-dimethylethylamine is obtained via the overhead as low boiler product and this is fed to a condenser in order to condense trimethylamine or N,N-dimethylethylamine. A portion of the condensed stream is fed back as return to the column and the remaining portion is fed back to the reactor (A) as recycle stream.

Therefore, a method is also preferred in which the reaction in step (a) is carried out in the presence of trimethylamine or N,N-dimethylethylamine as trialkylamine and methanol, and in distillation apparatus (B) in step (b)
(i) methanol is withdrawn as sidestream between the feed and the overhead, and
(ii) trimethylamine or N,N-dimethylethylamine as overhead product is condensed in a condenser, withdrawn from distillation apparatus (B) and is fed back to the reactor (A).

In a further preferred variant using trimethylamine or N,N-dimethylethylamine as trialkylamine, which are lower boiling than methanol, a dividing wall column is used in step (b). Trimethylamine or N,N-dimethylethylamine is obtained in this case via the overhead as low boiler product and this is fed to a condenser in order to condense trimethylamine or N,N-dimethylethylamine. A portion of the condensed stream is fed back as return to the column and the remaining portion is fed back to the reactor (A) as recycle stream. The desired amount of methanol as purge stream is withdrawn as sidestream.

In a preferred variant, using a trialkylamine such as triethylamine for example, which is higher boiling than methanol, the low boiler product removed from the column of step (b) is substantially completely condensed in a condenser, a portion of the condensed stream is discharged as purge stream, a further portion of the condensed stream is fed back to the column as return and the residual portion of the condensed stream is fed back to the reactor (A) as recycle stream.

In another preferred variant using a trialkylamine such as triethylamine for example, which is higher boiling than methanol, triethylamine is withdrawn as side-stream from the rectifying section of the column in step (b) and this is fed back to the reactor (A) as recycle stream. Methanol is obtained as top stream, which is condensed, the desired amount is discharged as purge stream and the remaining stream is fed back to the column as return.

In a further preferred variant using a trialkylamine such as triethylamine for example, which is higher boiling than methanol, a dividing wall column is used in step (b). Methanol is obtained in this case via the overhead as low boiler product and this is fed to a condenser in order to condense methanol. The desired amount is discharged from the condensate as purge stream and the remaining stream is fed back to the column as return. The trialkylamine which is higher boiling than methanol is withdrawn as sidestream and this is fed back to the reactor (A) as recycle stream.

In principle, although not preferred, in step (b) it is also possible to remove in one step also further low boilers, such as water and diethyl ketone (III) for example, or equally any components lower boiling than 1-hydroxy-2-methyl-3-pentanone (I). A stream highly enriched with 1-hydroxy-2-methyl-3-pentanone (I) would then be obtained as bottom product, which in addition still comprises diverse components higher boiling than 1-hydroxy-2-methyl-3-pentanone (I), such as oligomers or polymers of the enones for example. It is then possible, as required, to separate 1-hydroxy-2-methyl-3-pentanone (I) as low boiler from higher boiling components in a further distillation apparatus and thus to obtain a more purified form. The top product of the first distillation apparatus, which in addition to the alkylamine and optionally methanol still comprises further low boilers such as water and diethyl ketone (III) for example, can then be discarded for example or can be further distilled to recover individual components.

However, in step (b) in the method according to the invention, particular preference is given to removing trialkylamine and, if present in the reaction mixture, also methanol as low boilers, and to withdrawing water and diethyl ketone together with 1-hydroxy-2-methyl-3-pentanone (I) as high boilers.

The reaction in step (a) as well as the distillative separation in step (b) in the method according to the invention is preferably carried out continuously.

In the variant in which water and diethyl ketone (III) together with 1-hydroxy-2-methyl-3-pentanone (I) are withdrawn as high boilers in step (b), it is in turn particularly preferred, from the bottom product comprising 1-hydroxy-2-methyl-3-pentanone (I), to separate diethyl ketone (III) and water as low boilers and a bottom product comprising 1-hydroxy-2-methyl-3-pentanone (I) as high boiler in step (c) in a distillation apparatus (C).

In principle, any distillation apparatuses can be used as distillation apparatus (C) which are suitable for separating diethyl ketone and water from a mixture having a 3-hydroxyketone that is higher boiling than diethyl ketone. As for customary distillation apparatuses, in addition to the column body itself, distillation apparatus (C) naturally also has a device for heating the bottom of the column, typically referred to as bottoms reboiler, and also a condenser. The term distillation apparatus (C) is to be understood as the whole distillation unit including column body, bottom reboiler and condenser, as well as associated withdrawal lines and return lines. The column body typically comprises trays, structured packings or random packings, preferably structured packings. The number of theoretical plates is typically from 10 to 50, preferably≥15 and particularly preferably≥18, and also preferably≤30 and particularly preferably≤26, Generally, the feed is introduced in the middle region of the column body. The condensers used in the distillation apparatus (C) are especially air coolers as well as water-cooled condensers of any construction type, such as shell and tube or plate heat exchangers.

In principle, the distillation apparatus (C) can be operated at elevated pressure, at atmospheric pressure or reduced pressure. However, operation under reduced pressure is particularly advantageous at a top pressure of 0.001 to 0.09 MPa abs, particularly preferably at≥0.005 MPa abs, especially preferably at≥0.01 MPa abs and particularly at≥0.02 MPa abs, and also particularly preferably at≤0.05 MPa abs, especially preferably at≤0.04 MPa abs and particularly at≤0.03 MPa abs.

Since with increasing concentration of 1-hydroxy-2-methyl-3-pentanone (I), higher-boiling by-products such as the enone (VIII) for example or diverse enone dimers are also concentrated, and thus the risk of forming oligomers and polymers increases, it is advantageous to counteract the formation of higher molecular weight by-products. Therefore, a polymerization stabilizer is preferably fed to distillation apparatus (C). This can be supplied directly into the column body, in the feed stream out of distillation apparatus (B) or into the return stream of the reboiler or condenser. The addition is preferably via the feed stream out of the distillation apparatus (B). In principle, all stabilizers are suitable as possible stabilizers which counteract oligomerization and/or polymerization. Examples include phenothiazine, hydroquinone, 4-methoxyphenol, 4-hydroxy-2,2,6,6-tetramethylpiperidinyloxyl (“4-hydroxy-TEMPO”), 3,5-di-tert-butyl-4-hydroxytoluene and N,N′-di-sec-butyl-p-phenylenediamine. If a stabilizer is fed into the feed stream of distillation apparatus (C), the amount is typically 5 to 1000 ppm by weight, preferably≥10 ppm by weight and particularly preferably 20 ppm by weight, and also preferably≤500 ppm by weight and particularly preferably≤250 ppm by weight, based on the mass of 1-hydroxy-2-methyl-3-pentanone (I).

The stream comprising diethyl ketone (III), passed into the condenser in distillation apparatus (C) via the overhead, is condensed therein.

The low boiler product condensed in distillation apparatus (C) is now particularly preferably separated into an aqueous and an organic liquid phase, comprising diethyl ketone (III),
(i) organic liquid phase comprising diethyl ketone is withdrawn from distillation apparatus (C) and is fed back to the reactor (A); and
(ii) aqueous liquid phase is withdrawn from distillation apparatus (C).

A portion of the organic liquid phase comprising diethyl ketone (III) is in this case fed back to the column as column return. Typically, the distillation apparatus (C) is operated at a return ratio of 0.6 to 3, preferably of≥0.9 and particularly preferably of≥1, and also preferably of≤2 and particularly preferably of≤1.6.

Particularly preferably, 50 to 100% by weight and especially preferably 90 to 100% by weight, in particular the total amount of the organic liquid phase comprising diethyl ketone (III) which is not fed back to the column body as return is fed back to the reactor (A).

The separation in step (c) also preferred in the method according to the invention is preferably carried out continuously.

After water and diethyl ketone as low boilers and a bottom product comprising 1-hydroxy-2-methyl-3-pentanone (I) have been separated in the distillation apparatus (C) in step (c), it is advantageous, from the bottom product comprising 1-hydroxy-2-methyl-3-pentanone (I), to remove components that are higher boiling than 1-hydroxy-2-methyl-3-pentanone (I) as bottom product and to obtain 1-hydroxy-2-methyl-3-pentanone (I) as low boiler in a distillation apparatus (D).

In principle, any distillation apparatuses can be used as distillation apparatus (D), from which a β-hydroxyketone having a boiling point of about 200° C. (based on atmospheric pressure) can be separated from higher boiling β-hydroxyketones and enones. As for customary distillation apparatuses, in addition to the column body itself, distillation apparatus (D) naturally also has a device for heating the bottom of the column, typically referred to as bottoms reboiler, and also a condenser. The term distillation apparatus (D) is to be understood as the whole distillation unit including column body, bottom reboiler and condenser, as well as associated withdrawal lines and return lines. The column body typically comprises trays, structured packings or random packings, preferably structured packings. The number of theoretical plates is typically from 10 to 50, preferably≥15 and particularly preferably≥20, and also preferably≤40 and particularly preferably≤35. Generally, the feed is introduced in the middle region of the column body. The condensers used in the distillation apparatus (D) are especially air coolers as well as water-cooled condensers of any construction type, such as shell and tube or plate heat exchangers.

In principle, the distillation apparatus (D) can be operated at elevated pressure, at atmospheric pressure or reduced pressure. However, operation under reduced pressure is particularly advantageous at a top pressure of 0.0001 to 0.01 MPa abs, particularly preferably at≥0.0005 MPa abs, especially preferably at≥0.001 MPa abs and particularly at≥0.002 MPa abs, and also particularly preferably at≤0.005 MPa abs, especially preferably at≤0.004 MPa abs and particularly at≤0.003 MPa abs.

The stream comprising 1-hydroxy-2-methyl-3-pentanone (I) passed into the condenser in the distillation apparatus (D) via the overhead is condensed therein and a portion of the condensate is fed back to the column as column return. Typically, the distillation apparatus (D) is operated at a return ratio of 1 to 10, preferably of≥2 and particularly preferably of≥3, and also preferably of≤8 and particularly preferably of≤5. The distillate discharged from distillation apparatus (0) is purified 1-hydroxy-2-methyl-3-pentanone (I). The temperature in the condenser is preferably selected such that predominantly only 1-hydroxy-2-methyl-3-pentanone (I) condenses and possible residual amounts of lighter boiling components exit the condenser in gaseous form and are removed as gaseous top product from distillation apparatus (D).

The separation in step (d) also preferred in the method according to the invention is preferably carried out continuously.

From a process engineering perspective, it is therefore particularly advantageous to carry out both the reaction in step (a) and the subsequent distillative work-up continuously.

1-Hydroxy-2-methyl-3-pentanone (I) at a purity of over 90% by weight and preferably of over 94% by weight and a residual content of 2,4-dimethyl-5-hydroxy-1-penten-3-one (VIII) of≤3% by weight and preferably≤2.5% by weight can be obtained by the method according to the invention.

In a general embodiment of the method according to the invention for the continuous production of 1-hydroxy-2-methyl-3-pentanone (I), the reactants diethyl ketone (III) and formaldehyde (II) and also trialkylamine and water, which also may optionally originate completely from an aqueous formaldehyde solution, are fed continuously to a flow tube, which represent the reactor (A), at the desired pressure and the desired temperature. The reaction takes place therein. The geometry and the flow rate are assessed so that the residence time corresponds to the desired reaction duration. The reaction mixture obtained is then passed into the distillation apparatus (B) for removing the low boiler trialkylamine. By means of the top pressure of 0.2 to 1 MPa abs present therein, it is possible to cool the condenser without inconvenient refrigeration unit. An air cooler is preferably used. The trialkylamine removed from distillation apparatus (B) is preferably fed back again to the reactor (A).

In a preferred, further work-up of the bottom product comprising 1-hydroxy-2-methyl-3-pentanone (I) from distillation apparatus (B), this is fed to the separation of the low boilers water and diethyl ketone (III) in distillation apparatus (C). Distillation apparatus (C) is preferably operated under reduced pressure. The low boilers water and diethyl ketone (III) are condensed in the condenser and preferably fed to a separation device in which the condensate is separated into an aqueous and an organic liquid phase, comprising diethyl ketone (III). The diethyl ketone (III) removed from distillation apparatus (C) is preferably fed back again to the reactor (A). The aqueous phase is preferably removed from the system or fed back as water source to the reactor (A) to the extent required.

The bottom product comprising 1-hydroxy-2-methyl-3-pentanone (I) from distillation apparatus (C) is preferably fed to a distillation apparatus (D) to obtain purified 1-hydroxy-2-methyl-3-pentanone (I). This is also preferably operated under reduced pressure. 1-Hydroxy-2-methyl-3-pentanone (I) is then finally separated and removed as overhead product. High-boiling secondary components are obtained as bottom product. These are removed from the system and can be disposed of for example or used for thermal purposes.

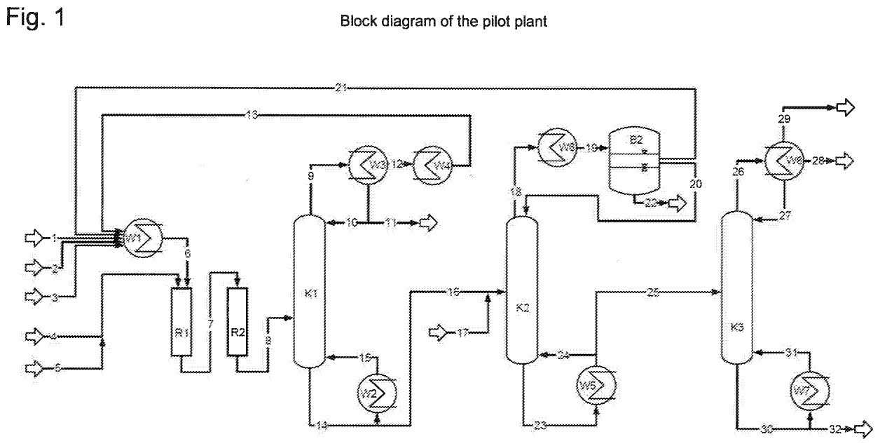

An exemplary representation of a suitable plant for carrying out a continuous process of this kind is shown in FIG. 1. A further description for this purpose is found below in the description of the pilot plant.

The method according to the invention enables the preparation of 1-hydroxy-2-methyl-3-pentanone (I) with high selectivity, yield and purity using the readily accessible feedstocks formaldehyde (II) and diethyl ketone (III), and reaction thereof in the presence of a likewise readily accessible trialkylamine. The method according to the invention is simple and safe to carry out. In particular, the method according to the invention is characterized in that the distillative removal of the trialkylamine from the reaction mixture obtained in the reaction can be effected without using costly refrigeration plants owing to the elevated pressure and elevated temperature in the distillation apparatus and nonetheless the formation of undesired by-products in the distillation apparatus specified increases only marginally. In particular, the amount of 2,4-dimethyl-5-hydroxy-1-penten-3-one (VIII), of which the boiling point is very close to the boiling point of 1-hydroxy-2-methyl-3-pentanone (I), and can therefore only by separated with great difficulty from 1-hydroxy-2-methyl-3-pentanone (I) by distillation, only increases marginally in the distillation apparatus mentioned in the method according to the invention.

By omitting a costly refrigeration plant, the method is therefore simpler in apparatus terms and more energy-efficient, both in regard to the apparatuses required and during operation.

### EXAMPLES

For the experiments which follow, a pilot plant and a laboratory-scale plant were available. Both plants are initally briefly described.

Pilot Plant

The simplified block diagram of the pilot plant is shown in FIG. 1. The individual apparatus labels have the following meanings therein:
W1: Heat exchanger (heated with thermostat)
R1, R2: Tubular reactors (reaction volumes 8.5 L each)
K1: Bubble-cap tray column (⊏55 mm, 45 trays)
W2: Falling-film evaporator (0.45 m², heated with thermostat)
W3: Condenser, 0.17 m² (cooled with cryostat)
W4: Condenser (0.16 m²)
K2: Column (□50 mm, Montz A3-750 packings, height 2560 mm)
W5: Falling-film evaporator (0.4 m², heated with thermostat)
W6: Condenser, (0.2 m², cooled with cryostat)
B2: Phase separator (0.15 L)
K3: Column (□43 mm, Montz A3-750 packings, height 2020 mm)
W7: Falling-film evaporator (0.4 m², heated with thermostat)
W8: Condenser, (0.2 m², cooled with cryostat)

Diethyl ketone (stream (1)), trimethylamine (stream (2)) and methanol (stream (3)) and the recycle streams (stream (13) and stream (21)) were fed to the heat exchanger W1 and subsequently passed into the tubular reactor R1 via stream (6). W1 served to adjust the inlet temperature of R1. In parallel thereto, the aqueous formaldehyde solution (stream (4)) and water (stream (5)) were passed into R1 together as separate streams. The reaction mixture from R1 was fed via stream (7) to tubular reactor R2. Both reactors were operated adiabatically. In the reactors, formaldehyde and diethyl ketone were reacted to give 1-hydroxy-2-methyl-3-pentanone (I) and diverse by-products.

The reaction mixture from R2 was fed via stream (8) to column K1 and trimethylamine and methanol were distilled off therein via the overhead. Column K1 is therefore simply referred to as “low boilers column”. In condenser W3, as much distillate was condensed so that in each case the return provided via stream (10) and the purge stream (11) required to set a constant methanol concentration was formed. In condenser W4, the residual vapors, which predominantly comprised trimethylamine, were condensed and fed back to the heat exchanger W1 via stream (13). In the falling-film evaporator W2, the temperature of the bottom stream was adjusted so that the methanol concentration in the bottom of column K1 was at most 0.1% by weight.

The bottoms output from column K1 was fed to column K2 as stream (16), wherein stabilizer (phenothiazine or hydroquinone) was metered in via stream (17). In column K2, unreacted diethyl ketone and water were distilled off via the overhead. Column K2 is therefore simply referred to as “diethyl ketone column”.

The vapor stream (18) formed was condensed in this case in condenser W6 and then passed into the phase separator B2. The condensate was separated therein into a more dense aqueous and a less dense organic phase. The aqueous phase was disposed of via stream (22). The organic phase, which comprised predominantly diethyl ketone, was passed back via stream (20) to adjust the desired return to column K2. The excess was fed back to the heat exchanger W1 via stream (21). In the falling-film evaporator W5, the temperature of the bottom stream was adjusted so that the diethyl ketone concentration in the outlet of falling-film evaporator W5 was approximately 0.1% by weight.

The bottoms output from column K2 was fed to column K3 via stream (25). In column K3, the 1-hydroxy-2-methyl-3-pentanone (I) formed and also enone (VII) was distilled off via the overhead. Column K3 is therefore simply referred to as “pure column”. The vapor stream (26) formed was subsequently condensed in condenser W8 by an appropriate temperature adjustment to the extent that 1-hydroxy-2-methyl-3-pentanone (I) could be withdrawn having only a low concentration of enone (VII) as stream (28). A portion of the condensate was fed back again to column K3 as return stream (27). The uncondensed enone (VII)-containing vapors were withdrawn via stream (29). In the falling-film evaporator W7, the temperature of the bottom stream was adjusted so that the concentration of enone (VIII) in the 1-hydroxy-2-methyl-3-pentanone (I)-containing product stream (28) was in the desired range. The high boilers separated were withdrawn via stream (32).

Laboratory Plant

A 300 mL thermostatically controllable steel autoclave served as laboratory-scale plant equipped with stirrer, thermocouple, pressure sensor and metered addition and withdrawing tube.

General Operation of the Pilot Plant

During operation of the pilot plant, the following fresh feeds were each fed. The further parameters are each described in the specific example.
Stream (1): Diethyl ketone (“DEK”)
Stream (2): Trimethylamine (“TMA”)
Stream (3): Methanol (“MeOH”)
Stream (4): aqueous formaldehyde solution (“FA solution”) having
   a formaldehyde content of 30% by weight (“FA30”) or 49% by weight
   (“FA49”) and in each case ca. 1% by weight methanol content
Stream (5): Water
Stream (17): Stabilizer solution (“Stabi solution”)
   Examples 1-3: Solution of 10% by weight phenothiazine in diethyl ketone
   Examples 4-7: Solution of 10% by weight hydroquinone in diethyl ketone

General Operation of the Laboratory-Scale Plant

To investigate the temperature stability of the bottom product of the pilot plant, 100 g of the corresponding bottom product was initially charged. The autoclave was sealed, purged with nitrogen, pressurized to 0.5 MPa abs nitrogen and heated to the desired temperature with stirring (900 rpm). The autoclave was further stirred at autogenous pressure and maintaining the desired temperature constant and, at certain time intervals, about 3-5 g samples were withdrawn in each case into a sample vial. The samples withdrawn were immediately cooled down in an ice bath in order to prevent further reaction and then intermediately stored in the refrigerator until analysis by gas chromatography. At the end of the experiment, the autoclave was cooled to room temperature, depressurized and emptied.

Quantitative Analysis

The quantitative analysis of the samples was conducted by gas chromatography (ZB-1 column, 30 m, 1 μm, 0.25 mm). In the case of a biphasic sample, this was firstly homogenized with ethanol and the ethanol peak subsequently deducted. Diethylene glycol diethyl ether served as internal standard for all quantitative GC analyses. By means of the response factors experimentally determined before-hand, methanol, diethyl ketone (III) and 1-hydroxy-2-methyl-3-pentanone (I) were determined quantitatively. For 1,5-dihydroxy-2,4-dimethyl-3-pentanone (IV), 1-hydroxy-2-hydroxymethyl-2-methyl-3-pentanone (V), 1, 5-dihydroxy-2-hydroxymethyl-2,4-dimethyl-3-pentanone (VI), 2-methyl-1-penten-3-one (VII) and 2,4-dimethyl-5-hydroxy-1-penten-3-one (VIII), the response factors were calculated on the basis of the “effective carbon number concept”.

### Example 1

Temperature Stability of the Bottom Product of Column K3

The bottom product of column K3 was produced for example 1 in the pilot plant under the operating conditions presented in Table 1 in column “Example 1” and withdrawn from the bottom of column K3. The bottom product withdrawn was analyzed by gas chromatography. Its content of 1-hydroxy-2-methyl-3-pentanone (I) and the by-products (IV) to (VIII) is presented in Table 2 under sampling time “0” hours.

In accordance with the description for the operation of the laboratory-scale plant, 100 g of the bottom product removed were transferred to the autoclaves and temperature-controlled therein as described. The temperature controlled in example 1 was 140° C. and the total experimental duration was 5 hours. The samples with-drawn a t certain time intervals were each analyzed by gas chromatography. The analytical data of the components
(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

are shown in table 2. Since Example 1 is the bottoms of pure column K3 and the components that are lighter boiling than 1-hydroxy-2-methyl-3-pentanone (I), such as trimethylamine, methanol, water and diethyl ketone (III) for example, have already been removed by columns 1 and 2, the bottoms of column K3 predominantly comprises the components that are higher boiling than 1-hydroxy-2-methyl-3-pentanone (I), such as components (IV) to (VIII) for example.

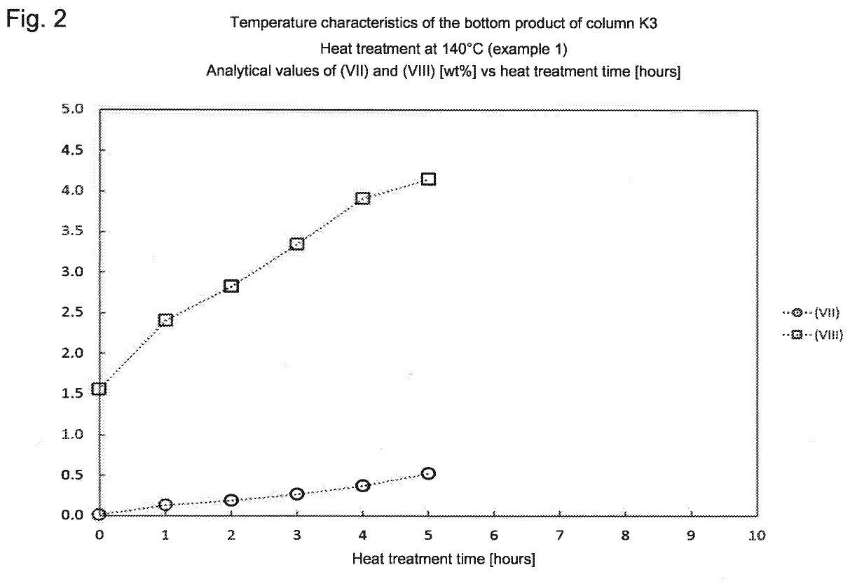

The analytical data show that, at a temperature controlled at 140° C., the enones (VII) and (VIII) formed by dehydration are greatly increased. FIG. 2 shows the increase of components (VII) and (VIII) over the heat treatment time in graphic form. For instance, the enone (VII) increase over 4 hours from 0.01 to 0.37% by weight, which corresponds to an increase of 3600%. By reason of its boiling point, the particularly disruptive enone (VIII) increases in the same time period from 1.56 to 3.91% by weight, which corresponds to an increase of 151%.

### Example 2

Temperature Stability of the Bottom Product of Column K1

The bottom product of column K1 was produced for example 2 in the pilot plant under the operating conditions presented in Table 1 in column “Example 2” and withdrawn from the bottom of column K1. The bottom product withdrawn was analyzed by gas chromatography. Its content of 1-hydroxy-2-methyl-3-pentanone (I) and the by-products (IV) to (VIII) is presented in Table 3 under sampling time “0” hours.

In accordance with the description for the operation of the laboratory-scale plant, 100 g of the bottom product removed were transferred to the autoclaves and temperature-controlled therein as described. The temperature controlled in example 2 was 140° C. and the total experimental duration was 10 hours. The samples withdrawn at certain time intervals were each analyzed by gas chromatography. The analytical data of the components
(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

are shown in table 3. Since example 2 is the bottoms of the low boiler column K1, this still comprises water and diethyl ketone (III) in addition to the target product 1-hydroxy-2-methyl-3-pentanone (I) and the by-products formed so far, such as the components (IV) to (VIII) for example. Therefore, in comparison to the bottom product of column K3, the relative content of 1-hydroxy-2-methyl-3-pentanone (I) is significantly higher and the relative content of components (IV) to (VIII) is in total significantly lower.

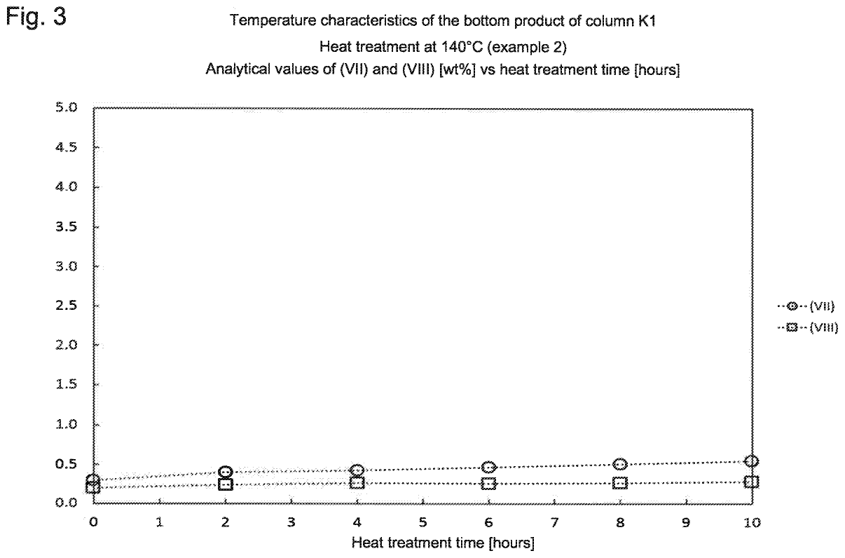

Despite a relatively high temperature control of 140° C., the enones (VII) and (VIII) formed by dehydration are surprisingly somewhat low compared to example 1. FIG. 3 shows the increase of components (VII) and (VIII) over the heat treatment time in graphic form. For instance, the enone (VII) only increases over 4 hours from 0.30 to 0.43% by weight, which corresponds to an increase of merely 43%. By reason of its boiling point, the particularly disruptive enone (VIII) increases in the same time period from 0.20 to 0.27% by weight, which only corresponds to an increase of 35%.

Table 4 summarizes the percentage changes of the by-products (IV) to (VIII) of examples 1 and 2.

Although in both examples the temperature was maintained at 140° C., the percentage increase in the disruptive by-products (VII) and (VIII) after 4 hours heat treatment time in example 2 is one to two orders of magnitude lower than that in example 1. This behaviour is completely surprising since an increase of by-products (VII) and (VIII) of approximately the same magnitude would have been expected in the case of identical thermal stress.

Examples 1 and 2 prove that column K1 can be operated also at a relatively high temperature without disproportionately increasing the formation of undesired enones (VII) and (VIII).

### Examples 3 to 7

Operation of Column K1 at Various Pressures

For this series of experiments, the pilot plant was operated under the operating conditions presented in Table 5. Each of the operating conditions set in examples 3 to 7 was maintained for 5 days in each case. The essential difference between the individual examples of this experimental series is the successively increased top pressure in column K1 from initially 0.1 MPa abs in example 3 to 0.525 MPa abs in example 7.

The top pressure in column K1 of only 0.1 MPa abs set in example 3 results in a relatively low bottom temperature of 87° C. and a relatively low condensation temperature in condenser W3 in the range of 18 to 26° C. The slightly varying top temperature over the 5 day experimental period is based on the slightly varying contents of trimethylamine and methanol in the vapor stream. The low condensation temperature for the condensation of the vapors in the heat exchangers W3 and W4 requires the use of a cooler cooling medium.

The higher top pressures in column K1 in examples 4 to 7 results in a higher condensation temperature in condenser W3 in the range of 60 to 87° C. As a result, it is possible to use a somewhat warmer temperature-controlled cooling medium than in example 3. Variations in the top temperature over the 5-day experimental procedure in each case is also here based on the slightly varying contents of trimethylamine and methanol in the vapor stream.

During the whole experimental series, a sample was taken from the bottom of column K1 and analyzed by gas chromatography. The contents determined of the components
(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

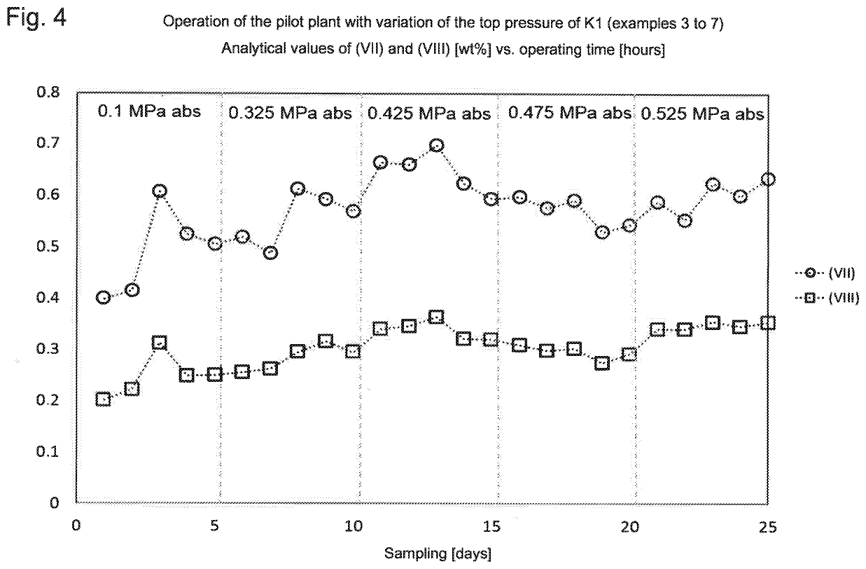

of the various samples are presented in Tables 6 to 10. FIG. 4 shows the time profile of components (VII) and (VIII) via the various settings, wherein the time scale here is presented continuously from day 1 to day 25.

Between a top pressure in column K1 of 0.1 MPa abs in example 3 and 0.525 MPa abs in example 7, although a certain increase of by-products (VII) and (VIII) can be observed, this is however against all expectation relatively low at about +45% for component (VII) and about +50% for component (VIM).

Examples 3 and 7 therefore show that a top pressure of 0.525 MPa abs itself in column K1 and the bottom temperature associated therewith of 138.8° C. only leads to a relatively low increase of by-products (VII) and (VIII). The resulting top temperature in the range of 82 to 96° C. employed for condensing the vapors would require milder cooling conditions than at a low top pressure of only 0.1 MPa abs.

The purified 1-hydroxy-2-methyl-3-pentanone (I) isolated from column K3 via stream 28 when carrying out examples 4 to 7 were analyzed for purity by gas chromatography. The values obtained for 1-hydroxy-2-methyl-3-pentanone (I) and component (VIII) are presented in Table 11. The measurement results show that even at a relatively high top pressure in column K1 of 0.525 MPa abs, 1-hydroxy-2-methyl-3-pentanone (I) was obtained at a high purity of 96.4% by weight and a relatively low content of component (VIII) of only 2.1% by weight.

TABLE 1
Operation of the pilot plant to obtain the bottom products of K3
and K1 at a temperature of 140° C. (examples 1 and 2)

| | | | | Example | |
|---|---|---|---|---|---|
| Apparatus | Description | Stream | Unit | 1 | 2 |
| | DEK | 1 | kg/h | 0.365 | 0.479 |
| | TMA | 2 | kg/h | 0.059 | 0.056 |
| | MeOH | 3 | kg/h | 0.071 | 0.028 |
| | FA | 4 | kg/h | 0.31 | 0.623 |
| | solution | | | (FA49) | (FA30) |
| | Water | 5 | kg/h | 0.249 | 0.041 |
| | TMA | 13 | kg/h | 0.51 | 0.49 |
| | Recycle | | | | |
| | DEK | 21 | kg/h | 1.66 | 1.56 |
| | Recycle | | | | |
| R1 | T(in) | 4 + 5 + 6 | ° C. | 93.4 | 83.9 |
| R2 | T(in) | 7 | ° C. | 84.8 | 88.9 |
| K1 | p(head) | 9 | MPa abs | 0.1025 | 0.1025 |
| | Return | 10 | kg/h | 2.9 | 3.2 |
| W2 | T | 15 | ° C. | 87.3 | 87.1 |
| K2 | p(head) | 18 | MPa abs | 0.0219 | 0.0219 |
| | Return | 20 | kg/h | 2.9 | 2.5 |
| | Stabi | 17 | kg/h | 0.001 | 0.001 |
| | solution | | | | |
| W5 | T | 24 | 00 | 140 | 140.9 |
| W6 | Condensate | 19 | ° C. | 10.2 | 32.8 |
| K3 | p(head) | 26 | MPa abs | 0.0028 | 0.0026 |
| | Return | 27 | kg/h | 1.6 | 1.6 |
| W7 | T | 31 | ° C. | 150 | 147 |
| W8 | T | 28 | ° C. | 45 | 71 |

TABLE 2
Temperature characteristics of the bottom product of column K3
at a temperature of 140° C. (example 1)

| Sampling | | | | | |
|---|---|---|---|---|---|
| time | (I) | (IV) + (V) | (VI) | (VII) | (VIII) |
| [hours] | [% by wt.] | [% by wt.] | [% by wt.] | [% by wt.] | [% by wt.] |
| 0 | 8.42 | 68.29 | 15.09 | 0.01 | 1.56 |
| 1 | 8.53 | 67.52 | 15.27 | 0.13 | 2.41 |
| 2 | 8.59 | 66.57 | 15.20 | 0.19 | 2.82 |
| 3 | 8.78 | 65.98 | 15.35 | 0.27 | 3.34 |
| 4 | 9.14 | 65.92 | 15.65 | 0.37 | 3.91 |
| 5 | 9.28 | 64.20 | 15.59 | 0.52 | 4.15 |

(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

TABLE 3
Temperature characteristics of the bottom product of column K1
at a temperature of 140° C. (example 2)

| Sampling | | | | | |
|---|---|---|---|---|---|
| time | (I) | (IV) + (V) | (VI) | (VII) | (VIII) |
| [hours] | [% by wt.] | [9.4 by wt] | [% by wt.] | [% by wt.] | [% by wt.] |
| 0 | 14.86 | 3.31 | 0.55 | 0.30 | 0.20 |
| 2 | 16.54 | 3.10 | 0.40 | 0.40 | 0.24 |
| 4 | 16.81 | 3.20 | 0.46 | 0.43 | 0.27 |
| 6 | 17.15 | 3.22 | 0.50 | 0.47 | 0.26 |
| 8 | 17.17 | 3.21 | 0.51 | 0.51 | 0.27 |
| 10 | 17.18 | 3.17 | 0.51 | 0.55 | 0.28 |

(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

TABLE 4
Change of the by-products in the bottom products of columns K3 and K1
by 4-hour temperature-control at 140° C. (examples 1 and 2)

| | Bottom product of column K3 | | | Bottom product of column K1 | | |
|---|---|---|---|---|---|---|
| | (Example 1) | | | (Example 2) | | |
| | Experiment | | | Experiment | | |
| | start | after | | start | after | |
| | (0 hours) | 4 hours | Percentage | (0 hours) | 4 hours | Percentage |
| By-product | [% by wt.] | [% by wt.] | Change | [% by wt.] | [% by wt.] | Change |
| (IV) + (V) | 68.29 | 65.92 | −3 | 3.31 | 3.20 | −3 |
| (VI) | 15.09 | 15.65 | +4 | 0.55 | 0.46 | −7 |
| (VII) | 0.01 | 0.37 | +3600 | 0.30 | 0.43 | +43 |
| (VIII) | 1.56 | 3.91 | +151 | 0.20 | 0.27 | +35 |

(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethy1-2,4-dimethyl-3-pentanone
(VII) 2-Methyl-1-penten-3-one
(VIII) 2,4-Dimethyl-5-hydroxy-1-penten-3-one

TABLE 5
Operation of the pilot plant under variation of the top pressure of K1

| | | | | Example | | | | |
|---|---|---|---|---|---|---|---|---|
| Apparatus | Description | Stream | Unit | 3 | 4 | 5 | 6 | 7 |
| | DEK | 1 | kg/h | 0.41 | 0.4 | 0.4 | 0.4 | 0.4 |
| | TMA | 2 | kg/h | 0.07 | 0.07 | 0.07 | 0.07 | 0.07 |
| | MeOH | 3 | kg/h | 0.031 | 0.016 | 0.016 | 0.016 | 0.016 |
| | FA solution | 4 | kg/h | 0.52 | 0.51 (FA30) | 0.51 (FA30) | 0.51 (FA30) | 0.51 (FA30) |
| | Water | 5 | kg/h | 0.051 | 0.052 | 0.052 | 0.052 | 0.052 |
| | TMA Recycle | 13 | kg/h | 0.49 | 0.49 | 0.49 | 0.49 | 0.49 |
| | DEK Recycle | 21 | kg/h | 1.65 | 1.65 | 1.65 | 1.65 | 1.65 |
| R1 | T(in) | 4 + 5 + 6 | ° C. | 84 | 84 | 91 | 90 | 93.5 |
| R2 | T(in) | 7 | ° C. | 88 | 86 | 89 | 90 | without R2 |
| K1 | p(head) | 9 | MPa abs | 0.1 | 0.325 | 0.425 | 0.475 | 0.525 |
| | T(top) | 9 | ° C. | 46-51 | 78-99 | 86-96 | 96 | 82-96 |
| | Return | 10 | kg/h | 3.2 | 3.2 | 3.2 | 3.2 | 3.2 |
| W2 | T(bottom) | 15 | ° C. | 87.1 | 121.9 | 131.1 | 134.9 | 138.8 |
| W3 | T | 12 | ° C. | 18-26 | 60-78 | 69-87 | 80-85 | 65-80 |
| K2 | p(head) | 18 | MPa abs | 0.0219 | 0.022 | 0.022 | 0.022 | 0.022 |
| | Return | 20 | kg/h | 2.5 | 2.5 | 2.5 | 2.5 | 2.5 |
| | Stabi solution | 17 | kg/h | 0.002 | 0.002 | 0.002 | 0.002 | 0.002 |
| W5 | T | 24 | ° C. | 140-141 | 141 | 141 | 141 | 141 |
| W6 | Condensate | 19 | ° C.. | 31-35 | 30 | 30 | 30 | 30 |
| K3 | p(head) | 26 | MPa abs | 0.0025- | 0.0025 | 0.0025 | 0.0025 | 0.0025 |
| | | | | 0.0029 | | | | |
| | Return | 27 | kg/h | 1.6 | 1.6 | 1.6 | 1.6 | 1.6 |
| W7 | T | 31 | ° C. | 145-150 | 145 | 145 | 143 | 145 |
| W8 | T | 28 | ° C. | 65-80 | 65.7 | 69.5 | 69.5 | 69.5 |

TABLE 6
Influence of the top pressure of column K1
on the composition of bottom product (example 3)

| p(top) K1 | T(bottom) K1 | Sampling | (I) | (IV) + (V) | (VI) | (VII) | (VIII) |
|---|---|---|---|---|---|---|---|
| [MPa abs] | [° C.] | after . . . days | [% by wt.] | [% by wt.] | [% by wt.] | [% by wt.] | [% by wt.] |
| 0.1 | 87.1 | 1 | 12.85 | 2.43 | 0.26 | 0.40 | 0.20 |
| | | 2 | 13.23 | 2.57 | 0.29 | 0.42 | 0.22 |
| | | 3 | 14.79 | 3.04 | 0.33 | 0.61 | 0.31 |
| | | 4 | 14.07 | 2.98 | 0.46 | 0.52 | 0.25 |
| | | 5 | 14.04 | 3.03 | 0.46 | 0.51 | 0.25 |

(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

TABLE 7
Influence of the top pressure of column K1
on the composition of bottom product (example 4)

| p(top) K1 | T(bottom) K1 | Sampling | (I) | (IV) + (V) | (VI) | (VII) | (VIII) |
|---|---|---|---|---|---|---|---|
| [MPa abs] | [° C.] | after . . . days | [% by wt.] | [% by wt.] | [% by wt.] | [% by wt.] | [% by wt.] |
| 0.325 | 121.9 | 1 | 12.68 | 2.41 | 0.29 | 0.52 | 0.26 |
| | | 2 | 13.85 | 2.78 | 0.32 | 0.49 | 0.26 |
| | | 3 | 14.19 | 2.77 | — | 0.61 | 0.30 |
| | | 4 | 14.30 | 2.94 | 0.40 | 0.59 | 0.32 |
| | | 5 | 14.09 | 2.81 | 0.36 | 0.57 | 0.30 |

(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

TABLE 8
Influence of the top pressure of column K1
on the composition of bottom product (example 5)

| p(top) K1 | T(bottom) K1 | Sampling | (I) | (IV) + (V) | (VI) | (VII) | (VIII) |
|---|---|---|---|---|---|---|---|
| [MPa abs] | [° C.] | after . . . days | [% by wt.] | [% by wt.] | [% by wt.] | [% by wt.] | [% by wt.] |
| 0.425 | 131.1 | 1 | 13.56 | 2.74 | 0.31 | 0.66 | 0.34 |
| | | 2 | 14.23 | 2.85 | 0.32 | 0.66 | 0.35 |
| | | 3 | 14.33 | 2.79 | 0.30 | 0.70 | 0.36 |
| | | 4 | 14.47 | 2.91 | 0.34 | 0.62 | 0.32 |
| | | 5 | 13.69 | 2.79 | 0.33 | 0.59 | 0.32 |

(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

TABLE 9
Influence of the top pressure of column K1
on the composition of bottom product (example 6)

| p(top) K1 | T(bottom) K1 | Sampling | (I) | (IV) + (V) | (VI) | (VII) | (VIII) |
|---|---|---|---|---|---|---|---|
| [MPa abs] | [° C.] | after . . . days | [% by wt.] | [% by wt.] | [% by wt.] | [% by wt.] | [% by wt.] |
| 0.475 | 134.9 | 1 | 14.00 | 2.80 | 0.31 | 0.60 | 0.31 |
| | | 2 | 13.68 | 2.78 | 0.32 | 0.58 | 0.30 |
| | | 3 | 13.90 | 2.81 | 0.32 | 0.59 | 0.30 |
| | | 4 | 12.62 | 2.58 | 0.30 | 0.53 | 0.27 |
| | | 5 | 13.76 | 2.82 | 0.33 | 0.54 | 0.29 |

(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

TABLE 10
Influence of the top pressure of column K1
on the composition of bottom product (example 7)

| p(top) K1 | T(bottom) K1 | Sampling | (I) | (IV) + (V) | (VI) | (VII) | (VIII) |
|---|---|---|---|---|---|---|---|
| [MPa abs] | [° C.] | after . . . days | [% by wt.] | [% by wt.] | [% by wt.] | [% by wt.] | [% by wt.] |
| 0.525 | 138.8 | 1 | 15.00 | 3.01 | — | 0.59 | 0.34 |
| | | 2 | 15.15 | 3.10 | 0.35 | 0.55 | 0.34 |
| | | 3 | 13.54 | 2.89 | 0.38 | 0.62 | 0.36 |
| | | 4 | 14.97 | 3.06 | 0.35 | 0.60 | 0.35 |
| | | 5 | 14.64 | 3.02 | 0.42 | 0.63 | 0.35 |

(I) 1-Hydroxy-2-methyl-3-pentanone
(IV) 1,5-Dihydroxy-2,4-dimethyl-3-pentanone
(V) 1-Hydroxy-2-hydroxymethyl-2-methyl-3-pentanone
(VI) 1,5-Dihydroxy-2-hydroxymethyl-2,4-dimethyl-3-pentanone
(VII) 2-Methyl-1-penten-3-one
(VIII) 2,4-Dimethyl-5-hydroxy-1-penten-3-one

TABLE 11
Purity of 1-hydroxy-2-methyl-3-pentanone (I) obtained via K3 (stream 28)
(Examples 4 to 7)

| | p(top) in K1 | (I) | (VIII) |
|---|---|---|---|
| Example | MPa abs | [% by wt.] | [% by wt.] |
| 4 | 0.325 | 95.1 | 2.5 |
| 5 | 0.425 | 94.9 | 2.6 |
| 6 | 0.475 | 95.7 | 2.3 |
| 7 | 0.525 | 96.4 | 2.1 |

(I) 1-Hydroxy-2-methyl-3-pentanone
(VIII) 2,4-Dimethyl-5-hydroxy-1-penten-3-one

1.-15. (canceled)

16. A method for preparing 1-hydroxy-2-methyl-3-pentanone (I) which comprises
(a) reacting formaldehyde (II) with diethyl ketone (III) in a reactor (A) in the presence of water and a basic component at a temperature of 50 to 150° C. and a pressure of 0.2 to 10 MPa abs,
wherein
the basic component used is a trialkylamine from the group comprising trimethylamine, N,N-dimethylethylamine, N,N-diethylmethylamine, triethylamine, N,N-dimethyl-n-propylamine, N-ethyl-N-methyl-n-propylamine, N,N-dimethylisopropylamine, N-ethyl-N-methylisopropylamine, N,N-dimethyl-n-butylamine, N,N-dimethylisobutylamine, N,N-dimethylsec-butylamine and
(b) from the reaction mixture obtained, trialkylamine as low boiler and a bottom product comprising 1-hydroxy-2-methyl-3-pentanone (I) as high boiler are separated in a distillation apparatus (B), wherein the distillation apparatus (B) is operated at a top pressure of 0.2 to 1 MPa abs.

17. The method according to claim 16, wherein
(c) from the bottom product of distillation apparatus (B) comprising 1-hydroxy-2-methyl-3-pentanone (I), diethyl ketone (III) and water as low boilers and a bottom product comprising 1-hydroxy-2-methyl-3-pentanone (I) as high boiler are separated in a distillation apparatus (C).

18. The method according to claim 17, wherein
(d) from the bottom product of distillation apparatus (C) comprising 1-hydroxy-2-methyl-3-pentanone (I), components higher boiling than 1-hydroxy-2-methyl-3-pentanone (I) are separated as bottom product and 1-hydroxy-2-methyl-3-pentanone (I) is obtained as low boiler.

19. The method according to claim 16, wherein the reaction is carried out at a molar ratio of diethyl ketone (III) to formaldehyde (II) in the liquid phase of from 1.5 to 10.

20. The method according to claim 16, wherein the trialkylamine used is trimethylamine or N,N-dimethylethylamine.

21. The method according to claim 16, wherein the reaction is carried out in the presence of 10 to 15% by weight water, based on the liquid phase.

22. The method according to claim 16, wherein the reaction is carried out in the presence of methanol.

23. The method according to claim 22, wherein the reaction is carried out in the presence of 0.1 to 10% by weight methanol, based on the liquid phase.

24. The method according to claim 16, wherein the distillation apparatus (B) reaction is operated at a top pressure of 0.2 to 0.6 MPa abs.

25. The method according to claim 16, wherein trialkylamine is condensed in a condenser in distillation apparatus (B), removed from the distillation apparatus (B) and fed back to the reactor (A).

26. The method according to claim 22, wherein methanol is also removed as low low boiler in distillation apparatus (B).

27. The method according to claim 16, wherein the reaction in step (a) is carried out in the presence of trimethylamine or N,N-dimethylethylamine as trialkylamine, and a two-stage condenser is used in distillation apparatus (B) for condensing the overhead product,
(i) in the first condenser, a mixture comprising methanol is condensed and withdrawn from distillation apparatus (B), and
(ii) in the second condenser, a mixture comprising trimethylamine or N,N-dimethylethylamine is condensed, withdrawn from distillation apparatus (B) and is fed back to the reactor (A).

28. The method according to claim 16, wherein the reaction in step (a) is carried out in the presence of trimethylamine or N,N-dimethylethylamine as trialkylamine and methanol and in distillation apparatus (B)
(i) methanol is withdrawn as sidestream between the feed and the overhead, and
(ii) trimethylamine or N,N-dimethylethylamine is condensed in a condenser as overhead product, withdrawn from distillation apparatus (B) and is fed back to the reactor (A).

29. The method according to claim 17, wherein low boiler product condensed in distillation apparatus (C) is separated into an aqueous and an organic liquid phase, comprising diethyl ketone (III),
(i) organic liquid phase comprising diethyl ketone (III) is withdrawn from distillation apparatus (C) and is fed back to the reactor (A) and
(ii) aqueous liquid phase is withdrawn from distillation apparatus (C).

30. The method according to claim 16, wherein the method is carried out continuously.
